(19) United States
(12) Patent Application Publication
(10) Pub. No.: US 2025/0067826 A1
(43) Pub. Date: Feb. 27, 2025

(54) RESONANCE-BASED PROPORTIONAL-INTEGRAL-DERIVATIVE (PID) CONTROL

(71) Applicant: FieldLine Inc., Boulder, CO (US)

(72) Inventors: Kenneth J. Hughes, Lafayette, CO (US); Tyler L. Maydew, Broomfield, CO (US)

(21) Appl. No.: 18/812,230

(22) Filed: Aug. 22, 2024

Related U.S. Application Data
(60) Provisional application No. 63/578,308, filed on Aug. 23, 2023.

(51) Int. Cl. G01R 33/54; A61B 5/00; A61B 5/245; G01R 33/48

(52) U.S. Cl. CPC G01R 33/543; A61B 5/245; A61B 5/6803; G01R 33/4806

(57) ABSTRACT

Various embodiments comprise a magnetic field detection system to control magnetometers. In some examples, the magnetic field detection system comprises a magnetometer controller. The magnetometer controller measures atomic resonance of a magnetometer. The magnetometer controller determines an error gain for the magnetometer based on the measured atomic resonance. The magnetometer controller applies the error gain to a measured error for the magnetometer and responsively calculates a control signal. The magnetometer controller adjusts the operation of the magnetometer based on the control signal.

### CROSS-REFERENCE TO RELATED APPLICATIONS

This U.S. Patent Application claims the benefit of and priority to U.S. Provisional Patent Application 63/578,308 titled, “RESONANCE-BASED PROPORTIONAL-INTEGRAL-DERIVATIVE (PID) CONTROL” which was filed on Aug. 23, 2023. U.S. Provisional Patent Application 63/578,308 is hereby incorporated by reference in its entirety into this U.S. Patent Application.

### TECHNICAL FIELD

Various embodiments of the present technology relate to control systems, and more specifically, to determining resonance-based error gains for Proportional-Integral-Derivative (PID) controllers.

### BACKGROUND

Control systems manage the behavior of other devices or systems based on control loops. In a control loop, the controlled system's output is compared to a setpoint for the controlled system. The resulting difference is defined as the error. The control system generates control signaling for the controlled system based on the error. The control signaling alters the operation of the controlled system to reduce the difference between the output and the setpoint. One type of control system is a Proportional-Integral-Derivative (PID) controller. A PID controller generates control signals based on a proportional term, an integral term, and a derivative term. The proportional term describes the overall difference between the setpoint and the measured error and is used to scale the output of the PID controller. When the difference between the setpoint and error is large, the proportional term is increased. Likewise, when the difference is small, the proportional term is decreased. The integral term accounts for past differences between the error and the setpoint. As the cumulative error between the setpoint and error decreases, so does the integral term. The derivative term accounts for the rate of change of the error. When the rate of change of the error increases, the derivative term increases. Likewise, when the rate of change of the error decreases, the derivative term decreases. The PID controller combines the proportional, integral, and derivative terms to generate a control signal which is then supplied to the controlled system.

PID controllers are used to control magnetometers. Magnetometers are devices that detect and characterize a magnetic field generated by a magnetic field source. Magnetometers measure the field strength and/or direction of the magnetic field to characterize the sensed field. Exemplary magnetometers include atomic magnetometers, Optically Pumped Magnetometers (OPMs), gradiometers, nitrogen vacancy centers, and high-temperature Superconducting Quantum Interference Devices (SQUIDs). Magnetometers may be used in anatomical magnetic sensing applications like Magnetoencephalography (MEG), Magnetocardiography (MCG), Magnetogastrography (MGG), and Magnetomyography (MMG). Some magnetometers are sensitive to fluctuations in the magnetic field and to changes in their operating parameters. For example, a small change in the operating parameters of the magnetometer may result in a large change in the magnetometer's output. This sensitivity increases the difficulty of implementing a PID control loop. In particular, conventional PID control systems often generate control signaling that overcorrects the operation of magnetometers.

Unfortunately, PID controllers do not efficiently control the operation of magnetometers. Moreover, PID controllers do not effectively account for device sensitivity when controlling magnetometers.

### OVERVIEW

This Overview is provided to introduce a selection of concepts in a simplified form that are further described below in the Technical Description. This summary is not intended to identify key features or essential features of the claimed subject matter, nor is it intended to be used as an aid in determining the scope of the claimed subject matter.

Various embodiments of the present technology relate to resonance-based control. Some embodiments comprise a method of operating a magnetic field detection system to control magnetometers. The method comprises measuring atomic resonance of a magnetometer. The method further comprises determining an error gain for the magnetometer based on the measured atomic resonance. The method further comprises applying the error gain to a measured error for the magnetometer and responsively calculating a control signal. The method further comprises adjusting the operation of the magnetometer based on the control signal.

Some embodiments comprise a magnetic field detection system to control magnetometers. The magnetic field detection system comprises a magnetometer controller. The magnetometer controller measures atomic resonance of a magnetometer. The magnetometer controller determines an error gain for the magnetometer based on the measured atomic resonance. The magnetometer controller applies the error gain to a measured error for the magnetometer and responsively calculates a control signal. The magnetometer controller adjusts the operation of the magnetometer based on the control signal.

Some embodiments comprise one or more non-transitory computer-readable storage media having program instructions stored thereon to control magnetometers. When executed by a computing system, the program instructions direct the computing system to perform operations. The operations comprise measuring atomic resonance of a magnetometer. The operations further comprise determining an error gain for the magnetometer based on the measured atomic resonance. The operations further comprise applying the error gain to a measured error for the magnetometer and responsively calculating a control signal. The operations further comprise adjusting the operation of the magnetometer based on the control signal.

### DESCRIPTION OF THE DRAWINGS

Many aspects of the disclosure can be better understood with reference to the following drawings. The components in the drawings are not necessarily drawn to scale. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates an exemplary resonance-based control system.

FIG. 2 illustrates an exemplary operation of the resonance-based control system.

FIG. 3 illustrates an exemplary magnetic field detection system.

FIG. 4 illustrates an exemplary operation of the magnetic field detection system.

FIG. 5 illustrates an exemplary operation of the magnetic field detection system.

FIG. 6 illustrates an exemplary Optically Pumped Magnetometer (OPM) Magnetoencephalography (MEG) system.

FIG. 7 illustrates an exemplary atomic resonance-based Proportional-Integral-

Derivative (PID) control system for an OPM.

FIG. 8 illustrates exemplary resonance curves for an OPM.

FIG. 9 illustrates exemplary resonance curves for an OPM.

FIG. 10 illustrates exemplary resonance curves for an OPM.

FIG. 11 illustrates exemplary resonance curves for an OPM.

FIG. 12 illustrates an exemplary computing apparatus for resonance-based control.

The drawings have not necessarily been drawn to scale. Similarly, some components or operations may not be separated into different blocks or combined into a single block for the purposes of discussion of some of the embodiments of the present technology. Moreover, while the technology is amendable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### DETAILED DESCRIPTION

The following description and associated figures teach the best mode of the invention. For the purpose of teaching inventive principles, some conventional aspects of the best mode may be simplified or omitted. The following claims specify the scope of the invention. Note that some aspects of the best mode may not fall within the scope of the invention as specified by the claims. Thus, those skilled in the art will appreciate variations from the best mode that fall within the scope of the invention. Those skilled in the art will appreciate that the features described below can be combined in various ways to form multiple variations of the invention. As a result, the invention is not limited to the specific examples described below, but only by the claims and their equivalents.

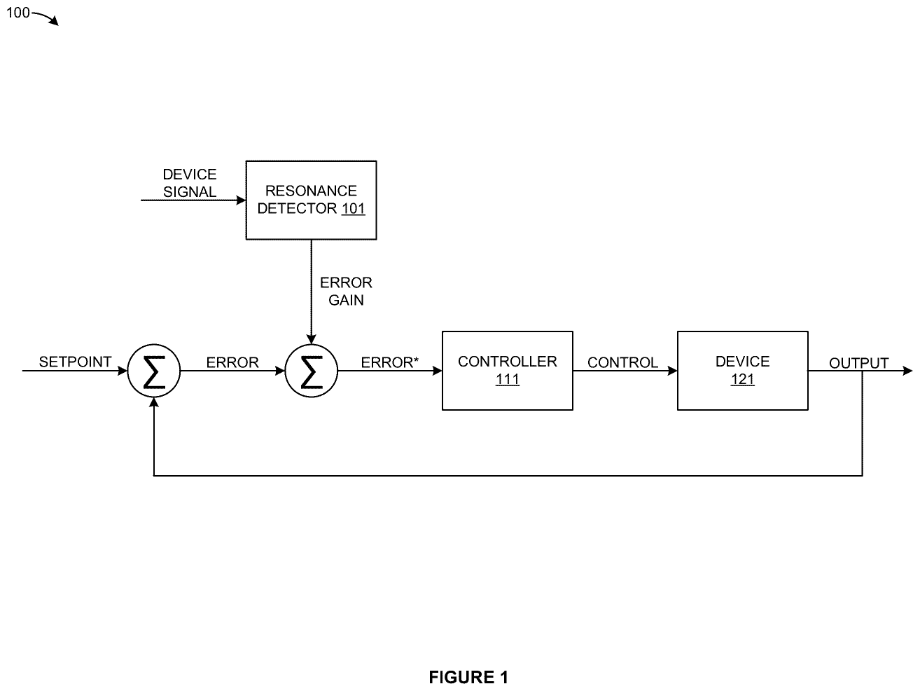

FIG. 1 illustrates resonance-based control system 100. Control system 100 implements a resonance-based control scheme to control the operation of a device. Control system 100 comprises resonance detector 101, controller 111, and device 121. In other examples, control system 100 may include fewer or additional components than those illustrated in FIG. 1. Likewise, the illustrated components of control system 100 may include fewer or additional components, assets, or connections than shown. Although resonance detector 101, controller 111, and device 121 are illustrated as separate devices, two or more of resonance detector 101, controller 111, and device 121 may be integrated into a single device and/or computing apparatus.

Resonance detector 101 is representative of a device or collection of devices to measure a resonance of device 121. Exemplary device types for detector 101 include computing devices, resonance detection electronics, and the like. The type of resonance measured by detector 101 depends in part on the device type of device 121. For example, the resonance may comprise an atomic resonance, mechanical resonance, electrical resonance, magnetic resonance, electromagnetic resonance, acoustic resonance, or some other type of resonance. Resonance detector 101 comprises one or more computing devices to determine the resonance of device 121 based on a signal generated by device 121. For example, the computing devices of detector 101 may receive a signal that depicts the resonance of device 121 and decode the signal to determine the resonance of device 121. Resonance detector 101 calculates a gain for the error signal of device 121 based on the measured resonance for device 121 and applies the gain to the error signal. The error signal is the difference between the setpoint and the output for device 121. For example, if the setpoint for device 121 comprises a temperature, the error signal for device 121 would be the difference between the setpoint temperature and the measured temperature of device 121. The gain-modified error signal is then delivered to controller 111. Resonance detector 101 may host a data structure, table, algorithm, and the like to translate the measured resonance into an error gain. The error gain scales the error signal based on the measured resonance to inhibit controller 111 from overcorrecting the operation of device 111.

Controller 111 is representative of one or more computing devices to control the operation of device 121. Exemplary controller types include Proportional-Integral-Derivative (PID) controllers, process controllers, or other types of devices that implement a control loop. Although the control loop illustrated in FIG. 1 comprises a feedback look, in other examples the control look may comprise a feedforward control loop. Controller 111 receives the gain adjusted error signal for device 121 and generates control signaling for device 121 based on the gain adjusted error. The control signaling adjusts the operation of device 121 to align output of device 121 with the setpoint of device 121. For example, controller 111 may comprise a PID controller that generates control signaling based on the gain-adjusted error signal and delivers the control signaling to device 121.

Device 121 is representative of any type of device or collection of devices that possesses a resonance. As is understood in the art, when an applied periodic force is equal to or close to the natural frequency of a system, the resulting amplitude of the of the system is greater than when the same periodic force is applied at some other frequency. The natural frequency of a system is the frequency the system tends to oscillate absent of any driving force. This increased amplitude is referred to as resonance. The frequency where the resonance occurs is referred to as the resonance frequency. Device 121 may comprise an atomic vapor cell, a magnetometer, a laser, an oscillating mechanical device, an oscillating acoustic device, or some other type of resonance system. Device 121 receives and implements control signaling for controller 111 to drive the operation of device 121 and account for any deviations between the setpoint and output of device 121. The type of resonator that composes device 121 is not limited. Accordingly, it should be appreciated that the type of resonance detection system implemented by detector 101 depends in part on the resonance type of device 121. For example, if device 121 possesses an optical resonance, resonance detector 101 may comprise optics and corresponding electronics to measure the optical resonance of device 121. For example, if device 121 possesses an atomic resonance, resonance detector 101 may comprise an atomic resonance detection system and corresponding electronics to measure the atomic resonance of device 121.

Resonance detector 101, controller 111, and device 121 communicate over various communication links. The communication links comprise metallic links, glass fibers, radio channels, or some other communication media. The links may use inter-processor communication, bus interfaces, Ethernet, WiFi, virtual switching, and/or some other communication protocol. Resonance detector 101, controller 111, and device 121 may comprise microprocessors, software, memories, transceivers, bus circuitry, and the like. The microprocessors comprise Central Processing Units (CPUs), Graphical Processing Units (GPUs), Digital Signal Processors (DSPs), Application-Specific Integrated Circuits (ASICs), Field Programmable Gate Arrays (FPGAs), analog computing circuits, and/or the like. The memories comprise Random Access Memory (RAM), flash circuitry, Hard Disk Drives (HDDs), Solid State Drives (SSDs), Non-Volatile Memory Express (NVMe) SSDs, and/or the like. The memories store software like operating systems, user applications, control applications, device applications, and the like. The microprocessors retrieve the software from the memories and execute the software to drive the operation of control system 100 as described herein.

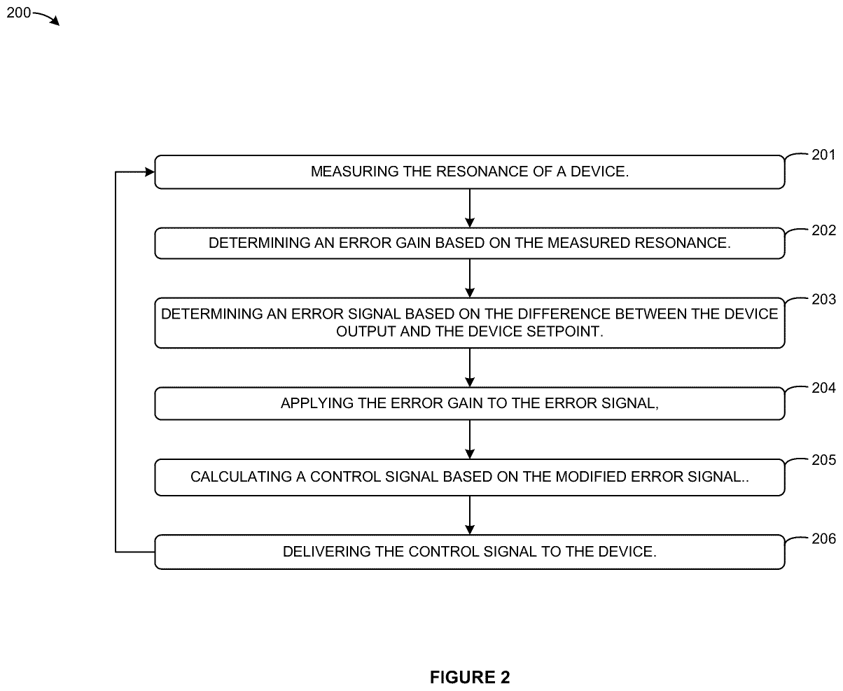

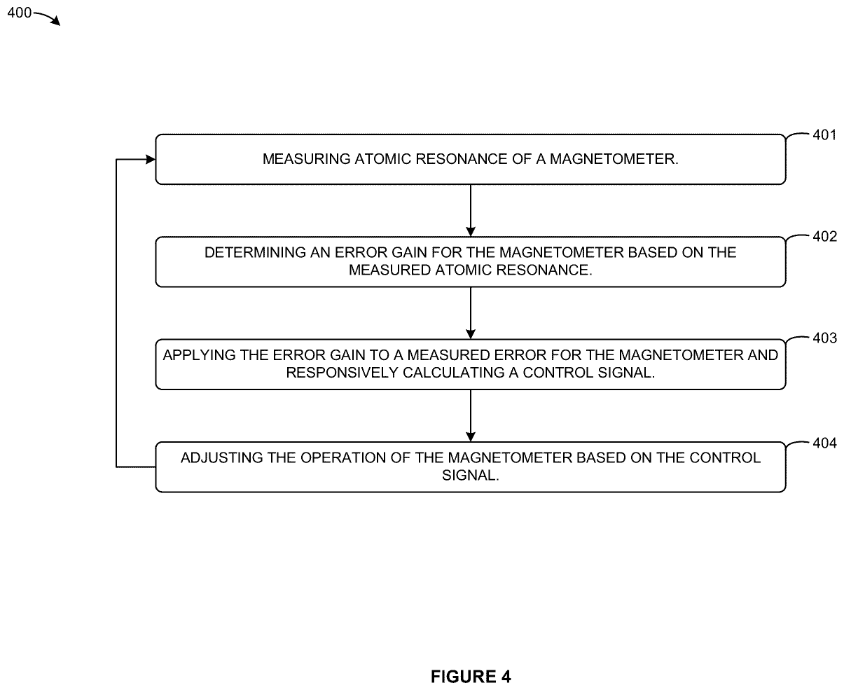

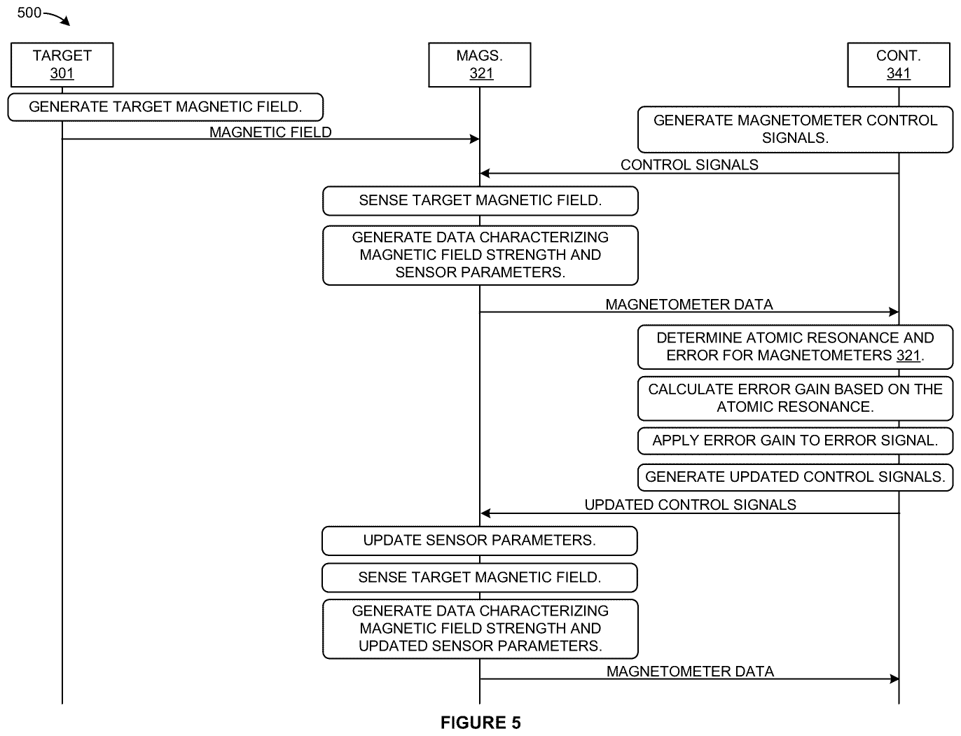

In some examples, control system 100 implements process 200 illustrated in FIG. 2, process 400 illustrated in FIG. 4, and/or process 500 illustrated in FIG. 5. It should be appreciated that the structure and operation of control system 100 may differ in other examples.

FIG. 2 illustrates process 200. Process 200 comprises a resonance-based control process. In other examples, process 200 may differ. Process 200 may be implemented in program instructions in the context of any of the software applications, module components, control components, resonance detection components, or other such elements of one or more computing devices. The program instructions direct the computing device(s) to operate as follows, referred to in the singular for the sake of clarity.

The operations of process 200 comprise measuring the resonance of a device (step 201). The operations further comprise determining an error gain based on the measured resonance (step 202). The operations further comprise determining an error signal based on the difference between the device output and the device setpoint (step 203). The operations further comprise applying the error gain to the error signal (step 204). The operations further comprise calculating a control signal based on the modified error signal (step 205). The operations further comprise delivering the control signal to the device (step 206). In some examples, process 200 repeats and the operations return to step 201 forming a feedback control loop.

Referring back to FIG. 1, control system 100 includes a brief example of process 200 as employed by one or more hardware components and software applications hosted by the various computing and electronic devices that compose control system 100.

In operation, an operator selects a setpoint for device 121. Controller 111 generates and transfers initial control signaling to drive the operation of device 121. Device 121 receives the control signaling and generates a system output and device signal. For example, in the case where device 121 comprises an atomic magnetometer, device 121 may output a device signal that characterizes photodetector absorption as a function of wavelength and may generate an output that characterizes measured magnetic field strength. Although illustrated as being separate, the device signal and the system output may comprise a single signal or may be the same.

Resonance detector 101 receives the device signal and measures the resonance of device 121 (step 201). For example, detector 101 may scan/sweep the device signal to identify the resonance frequency of device 121. Detector 101 correlates the measured resonance frequency to a gain for the error signal (step 202). Detector 101 may host a data structure, correlation table, or algorithmic processes to correlate resonance frequencies to error gains. For example, detector 101 may input the resonance for device 101 into an algorithm that calculates an error gain. For example, detector 101 may compare the resonance for device 101 to a table that associates device resonances to error gains and detector 101 may select one of the error gains from the table based on the comparison.

Resonance detector 101 compares the output of device 121 to the setpoint of device 121 and determines the error signal based on the difference (step 203). For example, the output for device 121 may comprise a magnetic field strength and detector 101 may determine the error signal for device 121 based on the difference between the measured magnetic field strength and the setpoint. Detector 121 applies the gain to the error signal and delivers the modified error signal to controller 111 (step 204). Controller 111 receives the modified error signal and responsively calculates a control signal for device 121 (step 205). Typically, applying the gain to the error signal involves adjusting coefficients used by controller 111 to calculate the control signaling. The error gain scales these coefficients to account for the measured resonance of device 121 to inhibit controller 111 from overcorrecting deviations between the output and setpoint of device 121. For example, when the measured resonance is high, the error gain may decrease the coefficients proportionally. Likewise, when the measured resonance is low, the error gain may increase the coefficients proportionally. Controller 111 delivers the control signal to device 121 (step 206). Device 121 updates its operating parameters based on the control signal to reduce the deviation between its output and setpoint.

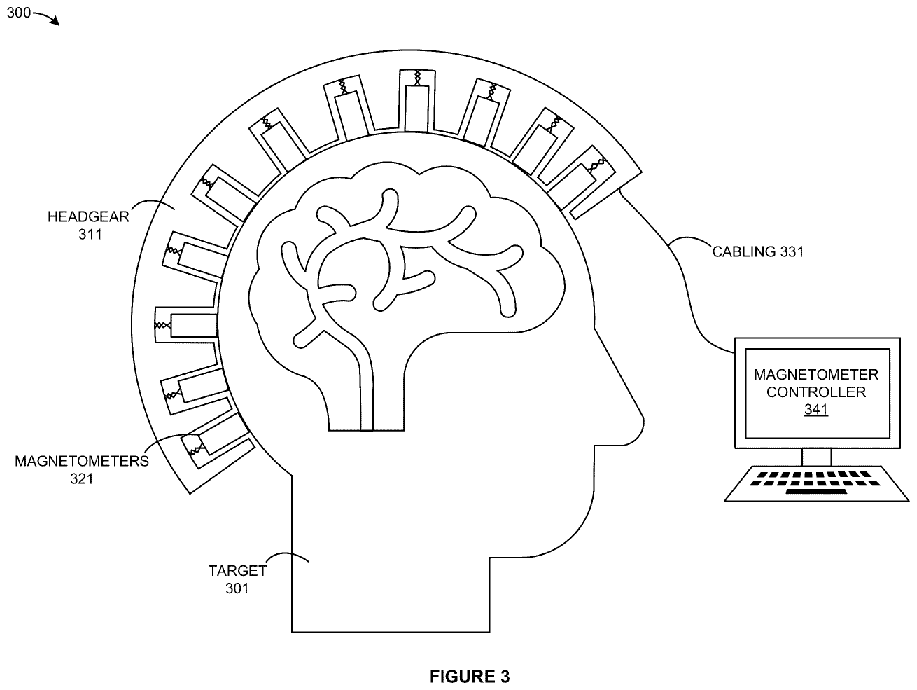

FIG. 3 illustrates magnetic field detection system 300. Magnetic field detection system 300 is an example of control system 100 illustrated in FIG. 1, however system 100 may differ. Magnetic field detection system 300 performs operations like detecting magnetic fields and relating the detecting magnetic fields to neuronal activity for use in medical applications. Exemplary medical applications include identifying brain activity and diagnosing conditions like stroke, epilepsy, brain injuries, brain disorders, and/or other types of medical conditions relating to brain/neuron activity. Magnetic field detection system 300 comprises target 301, headgear 311, magnetometers 321, cabling 331, and magnetometer controller 341. In other examples, magnetic field detection system 300 may differ. In this example, target 301 comprises a human head, however, target 301 may comprise any magnetic field source including a non-biological magnetic field source.

Various examples of system operation and configuration are described herein. In some examples, magnetometer controller 341 transfers instructions to magnetometers 321 over cabling 331. The instructions direct magnetometers 321 to measure a magnetic field generated by neuronal activity in target 301. Magnetometers 321 operate based on the instructions received from magnetometer controller 341 and sense the magnetic field generated by target 301. Magnetometers 321 transfer sensor data that characterizes the strength of the magnetic field and sensor operating parameters like photodetector intensity, vapor cell temperature, laser wavelength, and the like. Magnetometer controller 341 receives sensor data from magnetometers 321 that characterizes the strength of the sensed magnetic field. The sensor data may be addressed (e.g., sensor ID) to correlate the measured magnetic field strengths and sensor parameters with individual ones of magnetometers 321. Magnetometer controller 341 compares the sensed magnetic field strength to the sensor setpoint to determine a sensor error. Magnetometer controller 341 determines the atomic resonance for magnetometers 321 and an error gain based on the atomic resonance. Magnetometer controller 341 applies the gain to the sensor error and responsively generates control signaling for magnetometers 321. For example, magnetometer controller 341 may execute a PID application and input the gain adjusted error into the PID application to generate the control signaling. Magnetometer controller 341 transfers the control signaling to magnetometer 321 to adjust the operation of magnetometers 321. Magnetometers 321 adjust their operating parameters based on the control signaling to align their outputs with the setpoint. For example, the control signaling may drive magnetometer 321 to adjust laser wavelength, bias magnetic field strength, vapor cell temperature, and the like.

Headgear 311 comprises a wearable apparatus that secures the position and orientation of magnetometers 321 in locations proximate to target 301. For example, headgear 311 may securely adhere magnetometers 321 to the scalp of target 301. Headgear 311 comprises slots to hold magnetometers 321. The slots form channels that fix in place both the position and orientation of magnetometers 321. Headgear 311 is placed on the head of target 301 and magnetometers 321 move through their respective channels to contact the surface of target 301. Headgear 311 may comprise ratchet mechanisms, pneumatic mechanisms, set screws, springs, clamps, and/or another type of mechanism to move magnetometers 321 through the slots to contact target 301 and hold magnetometers 321 in place once in contact.

Magnetometers 321 comprise sensors that sense magnetic fields generated by a magnetic field source in target 301 and generate signals that characterize the strength of the detected magnetic field. In this example, the magnetic field source comprises the brain of target 301. The neuronal activity in the brain of target 301 comprises intercellular electromagnetic signals. Magnetometers 321 sense the magnetic component of the electromagnetic signals to detect neuronal activity. Magnetometers 321 may comprise atomic magnetometers, Optically Pumped Magnetometers (OPMs), gradiometers, nitrogen vacancy centers, high-temperature Superconducting Quantum Interference Devices (SQUIDs), and the like. Magnetometers 321 are coupled to controller 341 over cabling 331. Cabling 331 comprises sheathed metallic wires. For example, magnetometers 321 may transfer signaling that characterizes the sensed magnetic field and sensor operating parameters to magnetometer controller 341 over cabling 331. In some examples, cabling 331 may be replaced with, or used in addition with, a wireless transceiver system (e.g., antennas) to transfer communications between controller 341 and magnetometers 321 over a wireless networking protocol like Bluetooth.

Magnetometer controller 341 is representative of one or more computing devices configured to drive the operation of magnetometers 321 and to implement a resonance-based control loop to control magnetometers 321. The one or more computing devices comprise processors, memories, and transceivers that are connected over bus circuitry. The processors may comprise CPUs, GPUs, ASICs, FPGAs, and the like. The memories may comprise RAM, flash circuitry, SSDs, HDDs, NVMe SSDs, and the like. The memory stores software like operating systems, control applications, magnetometer applications, magnetometer data, and the like. The processors retrieve and execute the software from the memory to drive the operation of controller 341.

Although the above examples are discussed with relation to detecting magnetic fields that depict neuronal activity in the brain, other magnetic imaging modalities are contemplated herein. For example, magnetic field detection system 300 may comprise a Magnetoencephalography (MEG) system, Magnetocardiography (MCG) system, a Magnetogastrography (MGG) system, a Magnetomyography (MMG) system, or another type of anatomical magnetic sensing technology.

In some examples, magnetic field detection system 300 implements process 400 described in FIG. 4 and/or process 500 described in FIG. 5. It should be appreciated that the structure and operation of magnetic field detection system 300 may differ in other examples.

FIG. 4 illustrates process 400. Process 400 comprises a resonance-based control process. Process 400 comprises an example of process 200 illustrated in FIG. 2, however process 200 may differ. In other examples, process 400 may differ. Process 400 may be implemented in program instructions in the context of any of the software applications, module components, control components, resonance detection components, or other such elements of one or more computing devices. The program instructions direct the computing device(s) to operate as follows, referred to in the singular for the sake of clarity.

The operations of process 400 comprise measuring atomic resonance of a magnetometer (step 401). The operations further comprise determining an error gain for the magnetometer based on the measured atomic resonance (step 402). The operations further comprise applying the error gain to a measured error for the magnetometer and responsively calculating a control signal (step 403). The operations further comprise adjusting the operation of the magnetometer based on the control signal (404). In some examples, process 400 repeats and the operations return to step 401.

FIG. 5 illustrates process 500. Process 500 comprises an exemplary operation of magnetic field detection system 300 to implement an atomic resonance-based control loop for magnetometers 321. Process 500 comprises an example of process 200 illustrated in FIG. 2 and process 400 illustrated in FIG. 4, however processes 200 and 400 may differ. In operation, headgear 311 is placed on the head of target 301. Magnetometers (MAGS.) 321 are ratcheted down to contact the scalp of target 301. The neuronal activity in the brain of target 301 generates an electromagnetic field which passes through magnetometers 321.

Once magnetometers 321 are secured, magnetometer controller (CONT.) 341 generates and transfers control signaling to magnetometers 321. The control signaling directs magnetometers 321 to measure the magnetic component of the electromagnetic field emitted by target 301. For example, the control signaling 321 may select initial vapor cell temperatures, initial laser wavelengths, initial bias field strengths, and/or other operating parameters that govern the function of magnetometers 321. Magnetometers 321 receive the control signaling and sense the magnetic field generated by target 321. Magnetometers 321 generate data that characterizes the magnetic field strength and their sensor parameters. Magnetometers 321 transfers the magnetometer data to controller 341 over cabling 331.

Controller 341 processes the magnetometer data to determine error for magnetometers 321. For example, controller 341 may scan the magnetometer data using a lock in amplifier to determine the difference between the measured field strength and the setpoint field strength to determine the error for magnetometers 321. It should be appreciated that magnetometers 321 are directional. When the measurement orientation for magnetometers 321 differs from the direction of the target magnetic field, the measured field strength is lower than the actual field strength of the target magnetic field. Controller 341 identifies this difference as the error signal for magnetometers 321.

Controller 341 processes the magnetometer data to determine vapor cell atomic resonance for magnetometers 321. Controller 341 derives an error gain based on the atomic resonance. In this example, controller 341 hosts a function that algorithmically converts atomic resonance to error gain. Controller 341 inputs the atomic resonance into the function which outputs an error gain. Controller 341 applies the error gain to the measured error. The error gain scales the error signal based on the atomic resonance. As the atomic resonance increases, the error gain decreases the error signal proportionally. As the atomic resonance decreases, the error gain increases the error signal proportionally. By scaling the error signal, the error gain inhibits controller 341 from generating control signaling that overcorrects the operation of magnetometers 321. Controller 341 generates updated control signals based on the gain adjusted error. For example, controller 341 may input the gain adjusted error signal into a PID control function to generate the control signaling. Controller 341 transfers the updated control signals to magnetometers 321.

Magnetometers 321 receive the updated control signaling from controller 341 and adjust their operating parameters based on the control signaling. For example, magnetometers 321 may update their bias field strengths, laser wavelengths, vapor cell temperatures, or some other operating parameter of magnetometers 321. Magnetometers 321 sense the magnetic field generated by target 321. Magnetometers 321 generate subsequent data that characterizes the magnetic field strength and the updated sensor parameters. Magnetometers 321 transfer the subsequent magnetometer data to controller 341. Controller 341 may repeat the above-described resonance-based control process to further tune magnetometers 321.

Advantageously, magnetic field detection system 300 implements a resonance-based control process to efficiently control the operation of magnetometers 321. Moreover, magnetic field detection system 300 effectively adjusts measured errors for magnetometers 321 based on the atomic resonances for magnetometers 321. The gain-adjusted error signal inhibits controller 341 from generating control signaling that overcorrects the operation of magnetometers 321 to account for the sensitivity of magnetometers 321.

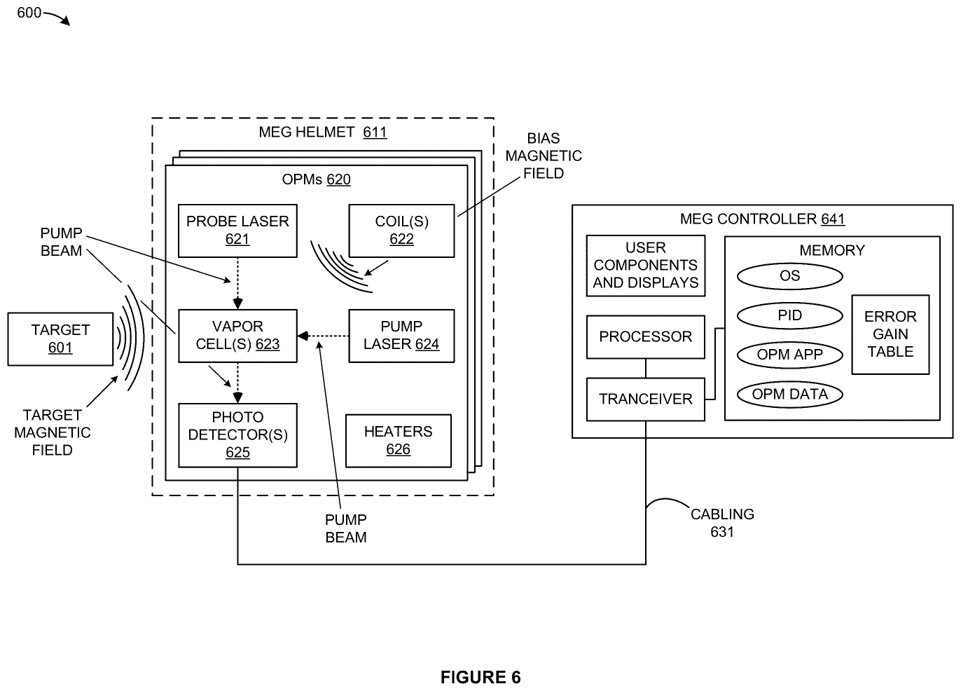

FIG. 6 illustrates OPM MEG system 600. OPM MEG system 600 comprises an example of control system 100 illustrated in FIG. 1 and magnetic field detection system 300 illustrated in FIG. 3, however systems 100 and 300 may differ. OPM MEG system 600 comprises target 601, MEG helmet 611, OPMs 620, cabling 631, and MEG controller 641. MEG helmet 611 mounts OPMs 620. In other examples, OPM MEG system 600 may comprise different or additional elements than those illustrated in FIG. 6.

Target 601 is magnetically linked to OPMs 620. OPMs 620 are metallically linked to cabling 631 which is metallically linked to the transceiver circuitry in MEG controller 641. Cabling 631 may be detachably coupled to MEG controller 641 and/or OPMs 620. MEG helmet 611 comprises a rigid helmet that holds OPMs 620 and is worn by target 601. MEG helmet 611 comprises ratchet mechanisms (not illustrated) to move OPMs 620 to contact the surface of target 601 when helmet 611 is worn by target 601. The ratchet mechanisms may comprise springs, pneumatics, electronic pistons, set screws, and the like. OPMs 620 each comprise probe laser 621, coil(s) 622, vapor cells 623, pump laser 624, photo detector(s) 625, and heaters 626. In some examples, the two lasers may be combined and/or additional lasers may be used. OPMs 620 typically comprise signal processors and other electronics, but they are omitted for sake of clarity. Controller 641 comprises transceiver circuitry, memory, a processor, user components, and displays. The processor comprises a CPU, GPU, DSP, FPGA, ASIC, and/or some other type of processing circuitry. The memory comprises RAM, HDD, SSD, NVMe SSD, and the like. The memories store software like operating systems (OS), and MEG applications for PID control, OPM applications, OPM data, and an error gain/resonance correlation table. The processor retrieves the software from the memory and executes the software to drive the operation of the MEG system 600 as described herein. The processor may write and read operational data to and from the memory. The operational data includes OPM Identifiers (IDs), magnetic field strengths, magnetic field directions, resonance frequencies, setpoints, configuration parameters, and OPM performance characteristics. In the following examples, OPMs 620 are referred to in the singular for the sake of clarity.

In operation, MEG helmet 611 is worn by target 601. The ratchet mechanism in helmet 611 moves OPM 620 until in contact with target 601. Once in contact with target 601, The processor in MEG controller 641 retrieves and executes the OPM application to generate control signals for OPM 620. The OPM application selects initial operating parameters like heater temperature, laser wavelength, and bias coil strength based on the OPM data stored in the memory. The OPM application generates instructions that direct OPM 620 to measure the magnetic field generated by target 601 and that include the operating parameters. The transceiver circuitry transfers the instructions to OPM 620.

OPM 620 operates in response to the instructions from MEG controller 641. Vapor cell 623 of OPM 620 is positioned in the target magnetic field. Vapor cell 623 contains an alkali metal like rubidium. Heaters 626 heat vapor cells 623 to vaporize the alkali metal and pressurize vapor cell 623. Coil 622 generates a bias magnetic field to orient the sensing direction of OPM 620 to align with the target magnetic field. OPM 620 is a directional magnetometer. The sensing direction of OPM 620 is typically oriented to measure a specific component (e.g., the normal component with respect to the surface of target 601) of the target magnetic field. As the orientation of OPM 620 changes with respect to the magnetic field direction, the measured strength for the target field component also changes.

Pump laser 624 emits a pump beam that is circularly polarized at a resonant frequency of the vapor contained by vapor cell 623 to polarize the atoms. Probe laser 621 emits a probe beam that is linearly polarized at a non-resonant frequency of the vapor to probe the atoms. The probe beam enters the vapor cells where quantum interactions with the atoms in the presence of the target magnetic field alter the energy/frequency of probe beam by amounts that correlate to the field strength of the target magnetic field. Photodetector 625 detects the probe beam after these alterations by the vapor atoms responsive to the magnetic field. Photodetector 625 generates corresponding analog electronic signals that characterize the measured field strength of the magnetic field, the probe/pump laser wavelengths, and the photodetector intensity. In some examples, a signal processor (not shown) may filter, amplify, digitize, or perform other tasks on the analog electronic signals. Photodetector 625 transfers an electronic signal that carries the data over cabling 631 to MEG controller 641.

MEG controller 641 processes the electronic signal received from OPM 620 to generate data that characterizes the measured field strength of the target magnetic field. The processor of MEG controller 641 retrieves and executes the PID application from memory. The PID application compares the measured field strengths at different orientations to the photodetector intensity to determine the measurement error for OPM 620. In particular, the PID application measures the difference between the measured field strength and the setpoint field strength to calculate the measurement error. For example, the PID application may plot the measured field strength for different measurement orientations as a function of photodetector intensity to compute a field strength curve. The PID application may then compute the derivative of this curve to determine the difference between the setpoint and the measured field strength. It should be appreciated that the measured field strength diminishes when the measurement orientation of OPM 620 differs from the setpoint orientation. The measurement orientation is governed in part by the strength of the bias magnetic field.

The PID application also compares the pump laser wavelengths to the reported photodetector intensity to determine the atomic resonance of vapor cell 623. For example, the PID application may plot the laser wavelengths as a function of photodetector intensity to compute an atomic resonance curve. The PID application may then compute the second derivative of the atomic resonance curve to derive the resonance frequency and resonance intensity of vapor cell 623. The PID application retrieves the error gain table from memory and compares the derived resonance frequency and intensity to the table. The error gain table lists resonance frequencies in correlation with error gains. The PID application matches the measured resonance frequency to one of the frequencies listed by the table and selects the corresponding error gain from the table. In other examples, controller 641 may instead host a function, algorithm, or some other type of data structure to correlate resonance frequencies to error gains.

The PID application applies the gain to the error for OPM 620. The gain scales the PID coefficients used by the PID application to account for the resonance frequency and intensity of vapor cell 623. Generally, as the resonance of vapor cell 623 increases, the PID coefficients are decreased proportionally. Likewise, as the resonance of vapor cell 623 decreases, the PID coefficients are increased proportionally. By scaling the coefficients, PID application is inhibited from generating control signaling that overcorrects the operation of OPM 620. For example, when the resonance of vapor cell is high, the resulting resonance curve is steep. Since the measured magnetic field strength is correlated to the resonance frequency, the field strength curve derived by controller 641 is also steep. As a consequence, small changes to the operating parameters of OPM 620 result in disproportionately large changes to the measured magnetic field strength. In this situation, the error gain dampens the error signal to inhibit the PID application from overcorrecting the operating parameters of OPM 620.

The PID application inputs the gain adjusted error for OPM 620 into its PID functions to generate a control output. The control output comprises instructions to adjust the bias field strength emitted by coil 622, the wavelength emitted by lasers 621 and/or 624, and/or the temperature of heaters 626. MEG controller 620 adjusts the bias field strength to account for deviations between measured field direction and the setpoint field direction. MEG controller 620 adjusts the laser wavelengths to account for deviations between applied laser wavelength and the resonance frequency wavelength. MEG controller 620 adjusts the temperature of heaters 626 to tune the cell pressure of vapor cell 623. The processor drives the transceiver circuitry to transfer the control output to OPM 620 over cabling 631.

OPM 620 receives the control output and adjusts the operation of lasers 621 and/or 624, coil 622, and/or heaters 626. In this example, the control output drives OPM 620 to adjust the wavelengths of lasers 621 and 624, the field strength of bias magnetic field 622, and the temperature of heaters 626. With the updated settings, coil 622 generates an updated bias magnetic field to change the sensing direction of OPM 620. Heaters 626 adjust the temperature of vapor cells 623. Pump laser 624 emits a pump beam at the updated frequency to polarize the atoms. Probe laser 621 emits a probe beam at the updated frequency to probe the atoms. Photodetector 625 detects the probe beam after these alterations by the vapor atoms responsive to the magnetic field. Photodetector 625 generates and transfers corresponding analog electronic signals that characterize the field strength, the probe laser wavelength, and the photodetector intensity to MEG controller 641. The PID application in controller 641 may repeat the control processes to further tune OPM 620 until the setpoint is achieved.

Once OPM 620 is tuned, MEG controller 641 executes the OPM application to generate a MEG image based on the magnetic field strengths received from OPM 620. The OPM application plots the magnetic field measurements in three dimensions based on the spatial locations of OPMs 620 with respect to target 601. The resulting MEG image depicts the magnetic field detected by OPMs 620 in three dimensions to illustrate the neuronal activity in the brain of target 601.

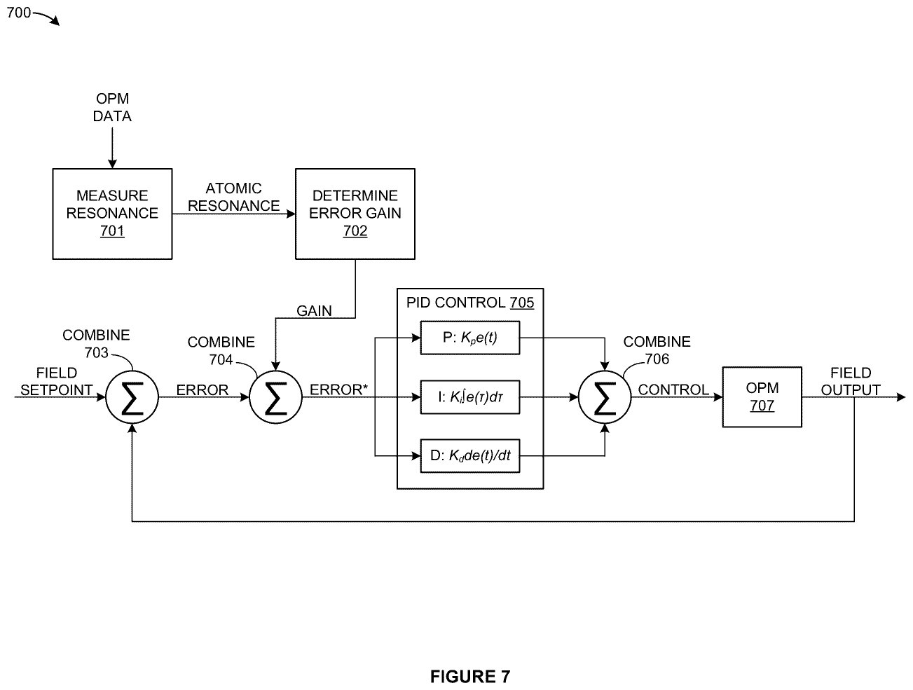

FIG. 7 illustrates atomic resonance-based PID control loop 700 for an OPM MEG system (e.g., OPM MEG system 600). Control loop 700 comprises an example of the control operations performed by the PID application described with respect to FIG. 6, however the operations of the PID application may differ. Control loop 700 is a block diagram and comprises measure resonance operation 701, determine error gain operation 702, combine operation 703, combine operation 704, PID control operation 705, combine operation 706, and OPM operation 707. In other examples, control the operations of control loop 700 may differ.

In some examples, a resonance detector scans OPM data to measure the resonance of an OPM vapor cell (operation 701). The OPM data comprises information that correlates OPM photodetector intensity to laser wavelengths. The resonance detector scans the OPM data to determine photodetector intensity at various laser frequencies to compute an atomic resonance curve for the vapor cell. The curve depicts the absorption for the OPM photodetector at different wavelengths and is typically semi-parabolic in shape. The resonance frequency corresponds to the wavelength where the photodetector absorption is at a minimum. To identify this minimum and the resonance intensity, the resonance detector takes the second derivative of the resonance curve and identifies the global maximum of the second derivative curve. The maximum of the second derivative curve corresponds to the resonance frequency and the resonance intensity. The resonance detector indicates the resonance frequency and resonance intensity to an error gain calculator.

The error gain calculator determines a gain for an error signal associated with the OPM (operation 702). In some examples, the gain calculator compares atomic resonance to a lookup table the correlates resonance frequencies to gain values. The gain calculator selects a resonance frequency listed by the table that most closely resembles the measured resonance frequency. In response, the gain calculator selects the gain that is correlated with the selected resonance frequency. In some examples, gain calculator comprises a function that converts resonance frequencies to gain values. The gain calculator inputs the resonance into the function. The function processes the resonance frequency to generate a gain value.

Contemporaneously, a lock in amplifier (or similar electronic device) scans target magnetic field strengths output by the OPM to determine the error signal. The field output comprises a signal that correlates OPM photodetector intensity to magnetic field measurement directions. The lock in amplifier determines OPM photodetector intensities at various OPM measurement orientations and computes a field strength curve based on the output. The curve depicts the absorption for the OPM photodetector at the different magnetic field measurement orientations and is typically semi-parabolic in shape. The setpoint for the OPM occurs at the global maximum of the field strength curve. To identify this maximum, the lock in amplifier takes the first derivative of the field strength curve and identifies the x-intercept of the resulting derivative curve. The lock in amplifier determines the difference between measurement orientation of the OPM and the maximum to determine the error signal.

A PID controller (or pre-processing system) applies the resonance-based gain determined by gain calculator to the error signal calculated by the lock in amplifier (operation 704). As illustrated in FIG. 7, the PID functions comprise:

$$P = {K}_{p} e ( t )$$

$$I = {K}_{i} \int e ( τ ) d τ$$

$$D = {K}_{d} \frac{de ( t )}{dt}$$

where P, I, and D are the proportional, integral, and derivative terms, Kₚ, Kᵢ, and K_{d} are the PID coefficients, and e(t) and e(τ) are the error values. The error gain scales the PID coefficients based on the measured resonance for the OPM vapor cell. As the vapor cell resonance increases, the PID coefficients are decreased proportionally. As the vapor cell resonance decreases, the PID coefficients are increased proportionally.

The PID controller calculates a proportional term (P), integral term (I), and derivative term (D) based on the error signal and gain modified coefficients (operation 705). The PID controller sums the terms to generate a control output and delivers the output to the OPM (operation 706). The control output typically adjusts the probe or pump laser wavelength to align the wavelength with the resonance frequency and/or adjusts the bias magnetic field strength to adjust the measurement orientation of the OPM to align the measurement orientation with the setpoint. The OPM receives the control signaling and adjusts its operating parameters accordingly to measure the target magnetic field (operation 707). Control loop 700 may repeat to continuously tune the operation of the OPM.

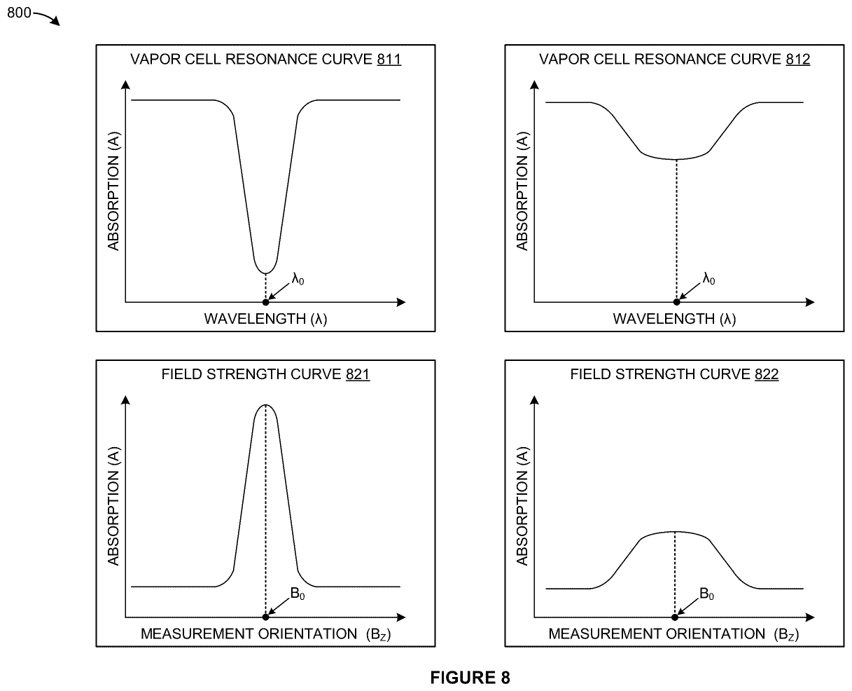

FIG. 8 illustrates environment 800. Environment 800 comprises vapor cell resonance curves 811 and 812 and field strength curves 821 and 822. Curves 811 and 812 depict the relationship between OPM photodetector absorption and laser wavelength. Curves 821 and 822 illustrate the relationship between OPM photodetector absorption and measurement orientation for an OPM. The λ₀ frequency comprises the resonance frequency for an OPM vapor cell and the B₀ comprises the OPM orientation setpoint. In this example, curves 811 and 821 correspond to a first OPM and curves 812 and 822 correspond to a second OPM.

As the measurement orientation of an OPM changes in the presence of a magnetic field, the photodetector intensity changes as depicted in curves 821 and 822. When the measurement orientation of the OMP converges on B₀, the photodetector absorption intensity increases. The photodetector intensity corresponds to a measured magnetic field strength. The measured magnetic field strength is maximized when orientation of the OPM is aligned with B₀.

In this example, field strength curve 821 corresponds to resonance curve 811 and field strength curve 822 corresponds to resonance curve 812. Curves 811 and 812 depict the resonance frequency for the two OPMs. When atomic resonance increases, the resonance curve increases in depth and decreases in width. Likewise, when the resonance decreases, the resonance decreases in depth and increases in width. As such, the atomic resonance for the first OPM depicted by curve 811 and is larger than the atomic resonance for the second OPM depicted by curve 812. The atomic resonance of an OPM vapor cell depends in part on the temperature and pressure of the vapor cell as well as other factors like Larmor presession and magnetic field gradients. Increasing atomic resonance intensity is advantageous. A high atomic resonance increases the magnetic field strength (e.g., photodetector intensity) measured by the OPM. As such, the field strength measurable by the first OPM as depicted by curve 821 is larger than the field strength measurable by the second OPM as depicted by curve 822.

It should be appreciated that slope of curve 821 is steeper than the slope of curve 822. Changes to the measurement orientation of the first OPM depicted by curve 821 result in a larger change to OPM photodetector absorption than to changes to the measurement orientation of the second OPM depicted by curve 822. When resonance is high, it can be difficult for a PID controller to align the measurement orientation of the OPM with B₀ due to this increase in sensitivity. As such, the resonance intensity depicted in curves 811 and 812 can be used to modify the error signal to alleviate this difficulty faced by the PID controller.

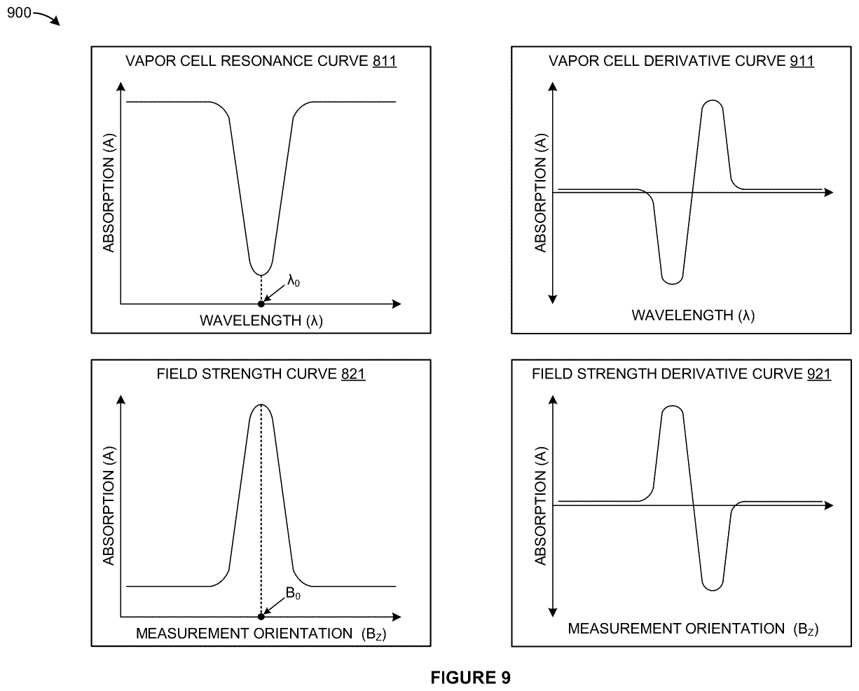

FIG. 9 illustrates environment 900. Environment 900 comprises resonance curves 811 and 821 and derivative curves 911 and 921. Derivative curve 911 depicts the first derivative of curve 811 and derivative curve 921 depicts the first derivative of curve 821. The x-intercept of derivative curve 911 corresponds to the resonance frequency λ₀. The x-intercept of derivative curve 921 corresponds to the orientation setpoint Bo. The slope of derivative curve 911 correlates to the size of the vapor cell resonance. Here, an increase in slope indicates an increase in vapor cell resonance while a decrease in slope indicates a decrease in vapor cell resonance. Field strength derivative curve 921 can be used to determine the error signal for a PID controller. For example, a lock in amplifier may scan photodetector absorptions and orientations output by the OPM to determine curve 821. The lock in amplifier may then take the derivative of curve 821 to generate curve 921. The lock in amplifier then determines the difference between the operating orientation reported by the OPM and the x-intercept of the derivative curve. The difference can then be used as the error signal for the PID controller.

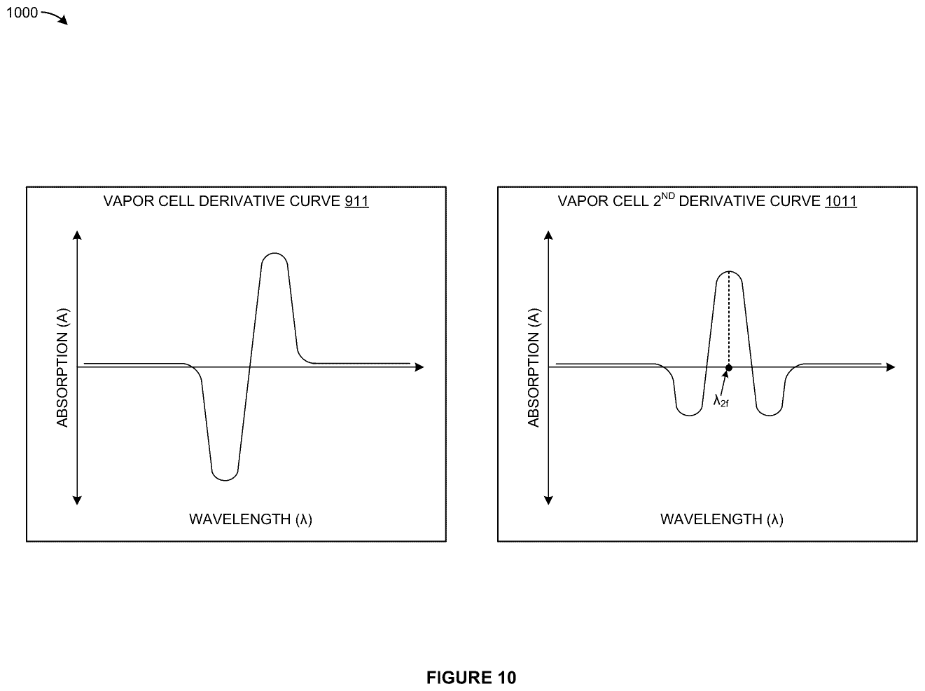

FIG. 10 illustrates environment 1000. Environment 1000 comprises vapor cell derivative curve 911 and vapor cell second derivative curve 1011. Vapor cell second derivative curve 1011 is the derivative of derivative curve 911 and the second derivative of vapor cell resonance curve 811. The peak (λ_{2f}) of vapor cell second derivative curve 1011 corresponds to the resonance frequency of the OPM vapor cell. The magnitude of the peak of second derivative curve 1011 correlates to the intensity of the resonance. A PID controller or pre-processing system can take the second derivative of curve 811 to form second derivative curve 1011. The PID controller identifies the global maximum of second derivative curve 1011 to identify the resonance frequency and determines the magnitude of the global maximum to determine the resonance intensity. When calculating the control signaling for the OPM, the PID scales its PID coefficients based on the magnitude of the resonance. When the resonance intensity is high, the scaling reduces the measured error (e.g., difference between OPM measurement orientation and B₀) proportionally. The scaling inhibits the PID controller from overcorrecting the measurement orientation of the OPM when the vapor cell resonance is high.

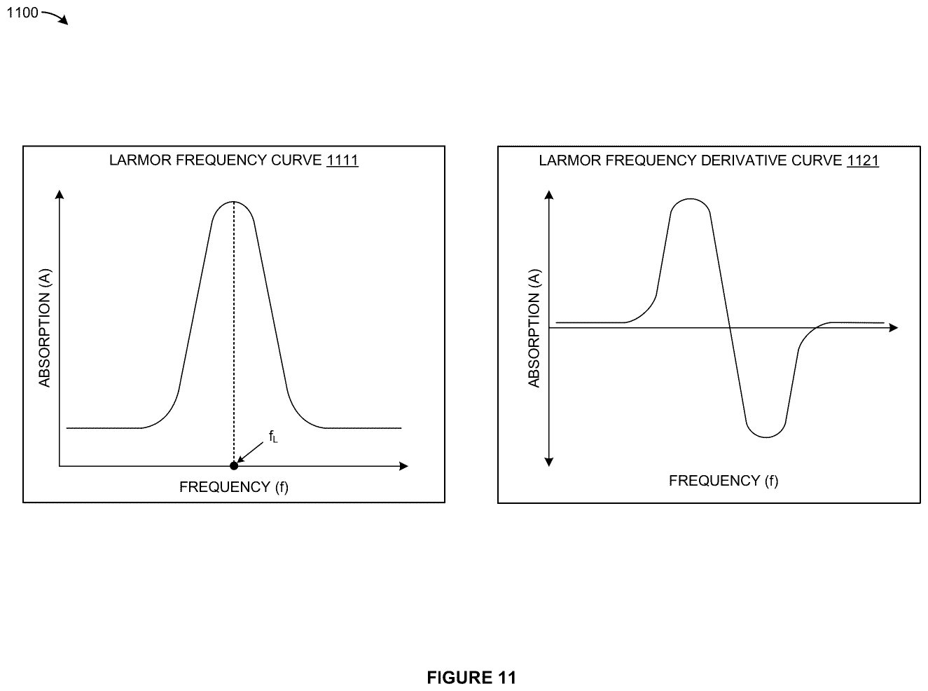

FIG. 11 illustrates environment 1100. Environment 1100 comprises Larmor frequency curve 1111 and Larmor frequency derivative curve 1121. The Larmor frequency describes the changing direction of the magnetic moment of an object in the presence of an external magnetic field and can affect the resonance of the OPM. An OPM may comprise a coil that applies a radio frequency to the atoms in the vapor cell to manipulate the Larmor precession. Larmor frequency curve 1111 illustrates the relationship between the Larmor frequency for the atoms and the photodetector absorption in the OPM. A PID controller may use Larmor derivative curve 1121 to determine the distance between and applied radio frequency and the Larmor resonance frequency (f_{L}). As illustrated in FIG. 11, the x-intercept of derivative curve 1121 corresponds to the Larmor frequency in curve 1111. The PID controller may determine the difference between the applied radio frequency and the Larmor resonance frequency and responsively adjust the radio frequency emitted by the OPM to target the Larmor resonance frequency. The PID controller may apply a gain to the error signal for the OPM based on the difference between the applied radio frequency and the Larmor resonance frequency. Targeting the Larmor frequency helps to maximize the resonance of the OPM vapor cell.

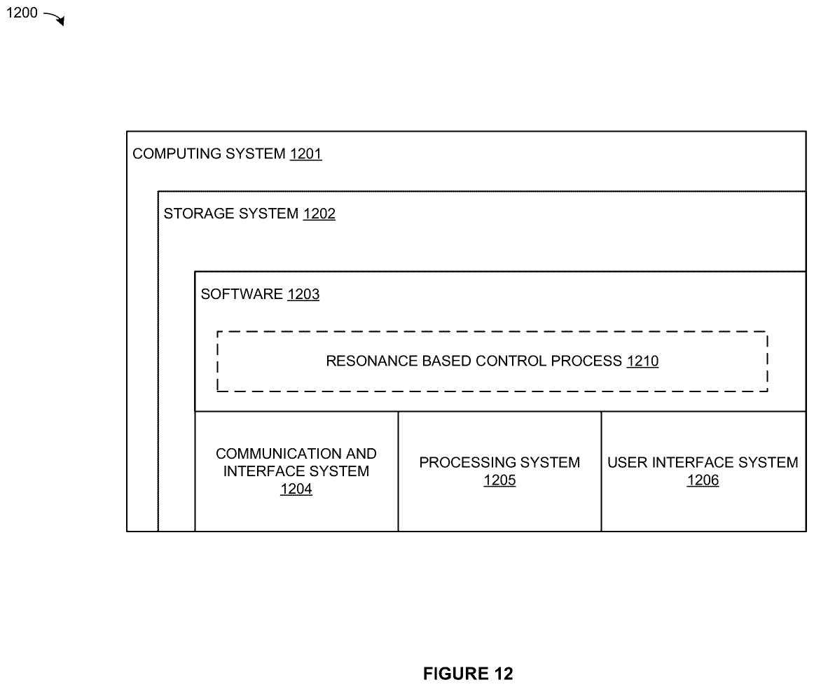

FIG. 12 illustrates computing environment 1200. Computing environment 1200 comprises computing system 1201. Computing system 1201 is representative of any system or collection of systems with which the various operational architectures, processes, scenarios, and sequences disclosed herein for performing resonance-based control. For example, computing system 1201 may be representative of resonance detector 101, controller 111, magnetometer controller 341, MEG controller 641, and/or any other computing device contemplated herein. Computing system 1201 may be implemented as a single apparatus, system, or device or may be implemented in a distributed manner as multiple apparatuses, systems, or devices. Computing system 1201 includes, but is not limited to, storage system 1202, software 1203, communication interface system 1204, processing system 1205, and user interface system 1206. Processing system 1205 is operatively coupled with storage system 1202, communication interface system 1204, and user interface system 1206.

Processing system 1205 loads and executes software 1203 from storage system 1202. Software 1203 includes and implements resonance-based control process 1210, which is representative of any of the resonance-based control processes described with respect to the preceding Figures, including but not limited to the resonance detection, error gain calculation, error signal adjustment, and control operations described with respect to the preceding Figures. For example, resonance-based control process 1210 may be representative of process 200 illustrated in FIG. 2, process 400 illustrated in FIG. 4, process 500 illustrated in FIG. 5, and/or control loop 700 illustrated in FIG. 7. When executed by processing system 1205 to implement a resonance-based control loop, software 1203 directs processing system 1205 to operate as described herein for at least the various processes, operational scenarios, and sequences discussed in the foregoing implementations. Computing system 1201 may optionally include additional devices, features, or functionality not discussed for purposes of brevity.

Processing system 1205 may comprise a micro-processor and other circuitry that retrieves and executes software 1203 from storage system 1202. Processing system 1205 may be implemented within a single processing device but may also be distributed across multiple processing devices or sub-systems that cooperate in executing program instructions. Examples of processing system 1205 include general purpose CPUs, GPUs, DSPs, ASICs, FPGAs, analog computing devices, and logic devices, as well as any other type of processing device, combinations, or variations thereof.

Storage system 1202 may comprise any computer readable storage media readable by processing system 1205 and capable of storing software 1203. Storage system 1202 may include volatile, nonvolatile, removable, and/or non-removable media implemented in any method or technology for storage of information, such as computer readable instructions, data structures, program modules, or other data. Examples of storage media include RAM, read only memory, magnetic disks, optical disks, optical media, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations storage system 1202 may also include computer readable communication media over which at least some of software 1203 may be communicated internally or externally. Storage system 1202 may be implemented as a single storage device but may also be implemented across multiple storage devices or sub-systems co-located or distributed relative to each other. Storage system 1202 may comprise additional elements, such as a controller, capable of communicating with processing system 1205 or possibly other systems.

Software 1203 (including resonance-based control process 1210) may be implemented in program instructions and among other functions may, when executed by processing system 1205, direct processing system 1205 to operate as described with respect to the various operational scenarios, sequences, and processes illustrated herein. For example, software 1203 may include program instructions for measuring atomic resonance of a magnetometer vapor cell, calculating an error gain based on the measured resonance, applying the gain to an error signal for the magnetometer, and generating control signaling for the magnetometer based on the gain adjusted error signal as described herein.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. Software 1203 may include additional processes, programs, or components, such as operating system software, virtualization software, or other application software. Software 1203 may also comprise firmware or some other form of machine-readable processing instructions executable by processing system 1205.

In general, software 1203 may, when loaded into processing system 1205 and executed, transform a suitable apparatus, system, or device (of which computing system 1201 is representative) overall from a general-purpose computing system into a special-purpose computing system customized to apply resonance-based control loop as described herein. Indeed, encoding software 1203 on storage system 1202 may transform the physical structure of storage system 1202. The specific transformation of the physical structure may depend on various factors in different implementations of this description. Examples of such factors may include, but are not limited to, the technology used to implement the storage media of storage system 1202 and whether the computer-storage media are characterized as primary or secondary storage, as well as other factors.

For example, if the computer readable storage media are implemented as semiconductor-based memory, software 1203 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 1204 may include communication connections and devices that allow for communication with other computing systems (not shown) over communication networks (not shown). Examples of connections and devices that together allow for inter-system communication may include network interface cards, antennas, power amplifiers, radiofrequency circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

Communication between computing system 1201 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of networks, or variation thereof. The aforementioned communication networks and protocols are well known and an extended discussion of them is omitted for the sake of brevity.

While some examples provided herein are described in the context of computing devices for resonance-based control processes, it should be understood that the control systems and methods described herein are not limited to such embodiments and may apply to a variety of other environments and their associated systems. As will be appreciated by one skilled in the art, aspects of the present invention may be embodied as a system, method, computer program product, and other configurable systems. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

These and other changes can be made to the technology in light of the above Detailed Description. While the above description describes certain examples of the technology, and describes the best mode contemplated, no matter how detailed the above appears in text, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the above Detailed Description section explicitly defines such terms. Accordingly, the actual scope of the technology encompasses not only the disclosed examples, but also all equivalent ways of practicing or implementing the technology under the claims.

What is claimed is:

1. A method of operating a magnetic field detection system to control magnetometers, the method comprising:
measuring atomic resonance of a magnetometer;
determining an error gain for the magnetometer based on the measured atomic resonance;
applying the error gain to a measured error for the magnetometer and responsively calculating a control signal; and
adjusting the operation of the magnetometer based on the control signal.

2. The method of claim 1 wherein measuring the atomic resonance of the magnetometer comprises determining an absorption curve for a magnetometer vapor cell.

3. The method of claim 2 wherein determining the error gain for the magnetometer based on the measured atomic resonance comprises determining a slope for the absorption curve and calculating the error gain based on the slope.

4. The method of claim 3 wherein calculating the error gain based on the slope comprises entering the slope into a data structure that algorithmically converts absorption curve slopes to error gains.

5. The method of claim 3 wherein calculating the error gain based on the slope comprises comparing the slope to a table that correlates absorption curve slopes to error gains.

6. The method of claim 1 wherein responsively calculating the control signal comprises calculating a Proportional-Integral-Derivative (PID) control signal based on the measured error modified by the error gain.

7. The method of claim 1 wherein adjusting the operation of the magnetometer based on the control signal comprises at least one of adjusting a laser wavelength, a bias magnetic field strength, or an operating temperature of the magnetometer.

8. A magnetic field detection system to control magnetometers, the magnetic field detection system comprising:
a magnetometer controller configured to:
measure atomic resonance of a magnetometer;
determine an error gain for the magnetometer based on the measured atomic resonance;
apply the error gain to a measured error for the magnetometer and responsively calculate a control signal; and
adjust the operation of the magnetometer based on the control signal.

9. The magnetic field detection system of claim 8 wherein the magnetometer controller is configured to determine an absorption curve for a magnetometer vapor cell.

10. The magnetic field detection system of claim 9 wherein the magnetometer controller is configured to determine a slope for the absorption curve and calculate the error gain based on the slope.

11. The magnetic field detection system of claim 10 wherein the magnetometer controller is configured to enter the slope into a data structure that algorithmically converts absorption curve slopes to error gains to calculate the error gain.

12. The magnetic field detection system of claim 10 wherein the magnetometer controller is configured to compare the slope to a table that correlates absorption curve slopes to error gains to calculate the error gain.

13. The magnetic field detection system of claim 8 wherein the magnetometer controller is configured to calculate a Proportional-Integral-Derivative (PID) control signal based on the measured error modified by the error gain.

14. The magnetic field detection system of claim 8 wherein the magnetometer controller is configured to adjust at least one of a laser wavelength, a bias magnetic field strength, or an operating temperature of the magnetometer.

15. One or more non-transitory computer-readable storage media having program instructions stored thereon to control magnetometers, wherein the program instructions, when executed by a computing system, direct the computing system to perform operations, the operations comprising:
measuring atomic resonance of a magnetometer;
determining an error gain for the magnetometer based on the measured atomic resonance;
applying the error gain to a measured error for the magnetometer and responsively calculating a control signal; and
adjusting the operation of the magnetometer based on the control signal.

16. The non-transitory computer-readable storage media of claim 15 wherein measuring the atomic resonance of the magnetometer comprises determining an absorption curve for a magnetometer vapor cell.

17. The non-transitory computer-readable storage media of claim 16 wherein determining the error gain for the magnetometer based on the measured atomic resonance comprises determining a slope for the absorption curve and calculating the error gain based on the slope.

18. The non-transitory computer-readable storage media of claim 17 wherein calculating the error gain based on the slope comprises entering the slope into a data structure that algorithmically converts absorption curve slopes to error gains.

19. The non-transitory computer-readable storage media of claim 17 wherein calculating the error gain based on the slope comprises comparing the slope to a table that correlates absorption curve slopes to error gains.

20. The non-transitory computer-readable storage media of claim 15 wherein:
responsively calculating the control signal comprises calculating a Proportional-Integral-Derivative (PID) control signal based on the measured error modified by the error gain; and
adjusting the operation of the magnetometer based on the control signal comprises at least one of adjusting a laser wavelength, a bias magnetic field strength, or an operating temperature of the magnetometer.
